(19) United States
(12) Patent Application Publication
(10) Pub. No.: US 2004/0259818 A1
(43) Pub. Date: Dec. 23, 2004

(54) Glucoronide adduct as gaba ligand

(76) Inventors: Byron Halevy, Watchung, NJ (US); Matthew Paul, Scotch Plains, NJ (US); Donghui Cui, Chalfont, PA (US); Stacey Lynn Polsky, Norristown (PA); A. David Rodrigues, Doylestown, PA (US); Jose Miguel Vega, Westlake Village, CA (US); Stanley Vickers, Penllyn, PA (US)

Correspondence Address: MERCK AND CO INC, P O BOX 2000, RAHWAY, NJ, 070650907

(21) Appl. No.: 10/477,025

(22) PCT Filed: May 3, 2002
(86) PCT No.: PCT/GB02/02075

(30) Foreign Application Priority Data
May 8, 2001 (GB) 0111191.3

(51) Int. Cl. A61K 31/7056; C07H 19/22

(52) U.S. Cl. 514/43; 536/27.1

(57) ABSTRACT

The quaternary ammonium N-glucuronide adduct of 7-(1,1-dimethylethyl)-6-(2-ethyl-2H-1,2,4-triazol-3-yl-methoxy)-3-(2-fluorophenyl)-1,2,4-triazolo[4,3-b]pyridazine is cleavable by glucuronidase enzymes in the body and can thereby act as a prodrug of a therapeutic agent which is a selective ligand for GABA_{A} receptors, in particular having high affinity for the α2 and/or α3 subunit thereof, and is accordingly of benefit in the treatment and/or prevention of disorders of the central nervous system, including anxiety and convulsions.

The present invention relates to the glucuronide adduct of a substituted triazolo-pyridazine derivative and to its use in therapy. More particularly, this invention is concerned with the covalent glucuronide adduct of a specific 1,2,4-triazolo[4,3-b]pyridazine derivative which is a GABA_{A} receptor ligand and is therefore useful in the therapy of deleterious mental states.

Receptors for the major inhibitory neurotransmitter, gamma-aminobutyric acid (GABA), are divided into two main classes: (1) GABA_{A} receptors, which are members of the ligand-gated ion channel superfamily; and (2) GABA_{B} receptors, which may be members of the G-protein linked receptor superfamily. Since the first cDNAs encoding individual GABA_{A} receptor subunits were cloned the number of known members of the mammalian family has grown to include at least six α subunits, four β subunits, three γ subunits, one δ subunit, one ε subunit and two ρ subunits.

Although knowledge of the diversity of the GABA_{A} receptor gene family represents a huge step forward in our understanding of this ligand-gated ion channel, insight into the extent of subtype diversity is still at an early stage. It has been indicated that an α subunit, a β subunit and a γ subunit constitute the minimum requirement for forming a fully functional GABA_{A} receptor expressed by transiently transfecting cDNAs into cells. As indicated above, 67, ε and ρ subunits also exist, but are present only to a minor extent in GABA_{A} receptor populations.

Studies of receptor size and visualisation by electron microscopy conclude that, like other members of the ligand-gated ion channel family, the native GABA_{A} receptor exists in pentameric form. The selection of at least one α, one β and one γ subunit from a repertoire of seventeen allows for the possible existence of more than 10,000 pentameric subunit combinations. Moreover, this calculation overlooks the additional permutations that would be possible if the arrangement of subunits around the ion channel had no constraints (i.e. there could be 120 possible variants for a receptor composed of five different subunits).

Receptor subtype assemblies which do exist include, amongst many others, α1β2γ2, α2β2/3γ2, α3βγ2/3, α2βγ1, α5β3γ2/3, α6βγ2, α6β≡ and α4βδ. Subtype assemblies containing an α1 subunit are present in most areas of the brain and are thought to account for over 40% of GABA_{A} receptors in the rat. Subtype assemblies containing α2 and α3 subunits respectively are thought to account for about 25% and 17% of GABA_{A} receptors in the rat. Subtype assemblies containing an α5 subunit are expressed predominantly in the hippocampus and cortex and are thought to represent about 4% of GABA_{A} receptors in the rat.

A characteristic property of all known GABA_{A} receptors is the presence of a number of modulatory sites, one of which is the benzodiazepine (BZ) binding site. The BZ binding site is the most explored of the GABA_{A} receptor modulatory sites, and is the site through which anxiolytic drugs such as diazepam and temazepam exert their effect. Before the cloning of the GABA_{A} receptor gene family, the benzodiazepine binding site was historically subdivided into two subtypes, BZ1 and BZ2, on the basis of radioligand binding studies. The BZ1 subtype has been shown to be pharmacologically equivalent to a GABA_{A} receptor comprising the α1 subunit in combination with a β subunit and γ2. This is the most abundant GABA_{A} receptor subtype, and is believed to represent almost half of all GABA_{A} receptors in the brain.

Two other major populations are the α2βγ2 and α3βγ2/3 subtypes. Together these constitute approximately a further 35% of the total GABA_{A} receptor repertoire. Pharmacologically this combination appears to be equivalent to the BZ2 subtype as defined previously by radioligand binding, although the BZ2 subtype may also include certain α5-containing subtype assemblies. The physiological role of these subtypes has hitherto been unclear because no sufficiently selective agonists or antagonists were known.

It is now believed that agents acting as BZ agonists at α1βγ2, α2βγ2 or α3βγ2 subtypes will possess desirable anxiolytic properties. Compounds which are modulators of the benzodiazepine binding site of the GABA_{A} receptor by acting as BZ agonists are referred to hereinafter as “GABA_{A} receptor agonists”. The α1-selective GABA_{A} receptor agonists alpidem and zolpidem are clinically prescribed as hypnotic agents, suggesting that at least some of the sedation associated with known anxiolytic drugs which act at the BZ1 binding site is mediated through GABA_{A} receptors containing the α1 subunit. Accordingly, it is considered that GABA_{A} receptor agonists which interact more favourably with the α2 and/or α3 subunit than with α1 will be effective in the treatment of anxiety with a reduced propensity to cause sedation. Also, agents which are antagonists or inverse agonists at α1 might be employed to reverse sedation or hypnosis caused by α1 agonists.

Compounds which are selective ligands for GABA_{A} receptors are of use in the treatment and/or prevention of a variety of disorders of the central nervous system. Such disorders include anxiety disorders, such as panic disorder with or without agoraphobia, agoraphobia without history of panic disorder, animal and other phobias including social phobias, obsessive-compulsive disorder, stress disorders including post-traumatic and acute stress disorder, and generalized or substance-induced anxiety disorder; neuroses; convulsions; migraine; depressive or bipolar disorders, for example single-episode or recurrent major depressive disorder, dysthymic disorder, bipolar I and bipolar II manic disorders, and cyclothymic disorder; psychotic disorders including schizophrenia; neurodegeneration arising from cerebral ischemia; attention deficit hyperactivity disorder; speech disorders, including stuttering; and disorders of circadian rhythm, e.g. in subjects suffering from the effects of jet lag or shift work.

Further disorders for which selective ligands for GABA_{A} receptors may be of benefit include pain and nociception; emesis, including acute, delayed and anticipatory emesis, in particular emesis induced by chemotherapy or radiation, as well as motion sickness, and post-operative nausea and vomiting; eating disorders including anorexia nervosa and bulimia nervosa; premenstrual syndrome; muscle spasm or spasticity, e.g. in paraplegic patients; and hearing disorders, including tinnitus and age-related hearing impairment. Selective ligands for GABA_{A} receptors may also be effective as pre-medication prior to anaesthesia or minor procedures such as endoscopy, including gastric endoscopy.

The present invention relates to the quaternary ammonium N-glucuronide adduct of a particular 1,2,4-triazolo[4,3-b]pyridazine derivative which, because it is capable of being cleaved by glucuronidase enzymes in the body, can thereby act as a prodrug thereof.

Specifically, the present invention provides the quaternary ammonium N-glucuronide adduct of 7-(1,1-dimethylethyl)-6-(2-ethyl-2H-1,2,4-triazol-3-ylmethoxy)-3-(2-fluorophenyl)-1,2,4-triazolo[4,3-b]pyridazine whose chemical structure is depicted in formula I:

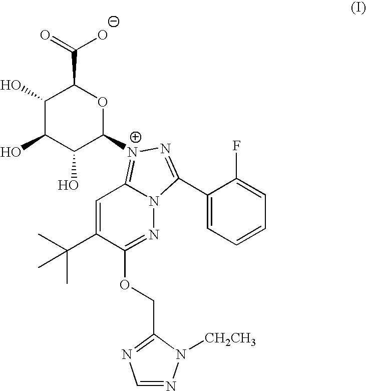

WO 99/67245 discloses 7-(1,1-dimethylethyl)-6-(2-ethyl-2H-1,2,4-triazol-3-ylmethoxy)-3-(2-fluorophenyl)-1,2,4-triazolo[4,3-b]pyridazine, and pharmaceutically acceptable salts thereof, which are stated to be selective ligands for GABA_{A} receptors, in particular having high affinity for the α2 and/or α3 subunit thereof, and hence beneficial in the treatment and/or prevention of neurological disorders including anxiety and convulsions. The disclosure of WO 99/67245 is encompassed within the generic scope of WO 98/04559, which describes a class of substituted and 7,8-ring fused 1,2,4-triazolo[4,3-b]pyridazine derivatives, including salts and prodrugs thereof. There is, however, no specific disclosure either in WO 99/67245 or in WO 98/04559 of the covalent glucuronide prodrug of formula I as depicted above.

By virtue of being the prodrug of an active triazolo-pyridazine derivative, the glucuronide adduct of the present invention possesses desirable pharmacological properties. The triazolo-pyridazine derivative of the present invention possesses advantageous binding properties at various GABA_{A} receptor subtypes, in particular having good affinity as a ligand for the α2 and/or α3 subunit of the human GABA_{A} receptor. The triazolo-pyridazine derivative of this invention interacts more favourably with the α2 and/or α3 subunit than with the α1 subunit. Indeed, the triazolo-pyridazine derivative of the invention exhibits functional selectivity in terms of a selective efficacy for the α2 and/or α3 subunit relative to the α1 subunit.

The triazolo-pyridazine derivative of the present invention is a GABA_{A} receptor subtype ligand having a binding affinity (Kᵢ) for the α2 and/or α3 subunit, as measured in the assay described hereinbelow, of less than 1 nM. Furthermore, the triazolo-pyridazine derivative of this invention exhibits functional selectivity in terms of a selective efficacy for the α2 and/or α3 subunit relative to the α1 subunit. Moreover, the triazolo-pyridazine derivative of the present invention possesses interesting pharmacokinetic properties, notably in terms of improved oral bioavailability.

Also provided by the present invention is a method for the treatment and/or prevention of anxiety which comprises administering to a patient in need of such treatment an effective amount of the compound of formula I as depicted above.

Further provided by the present invention is a method for the treatment and/or prevention of convulsions (e.g. in a patient suffering from epilepsy or a related disorder) which comprises administering to a patient in need of such treatment an effective amount of the compound of formula I as depicted above.

The binding affinity (Kᵢ) of the triazolo-pyridazine derivative of the present invention for the α3 subunit of the human GABA_{A} receptor is conveniently as measured in the assay described hereinbelow. The α3 subunit binding affinity (Kᵢ) of the triazolo-pyridazine derivative of the invention is less than 1 nM.

The triazolo-pyridazine derivative of the present invention elicits a selective potentiation of the GABA EC₂₀ response in stably transfected recombinant cell lines expressing the α3 subunit of the human GABA_{A} receptor relative to the potentiation of the GABA EC₂₀ response elicited in stably transfected recombinant cell lines expressing the α1 subunit of the human GABA_{A} receptor.

The potentiation of the GABA EC₂₀ response in stably transfected cell lines expressing the α3 and α1 subunits of the human GABA_{A} receptor can conveniently be measured by procedures analogous to the protocol described in Wafford et al., Mol. Pharmacol., 1996, 50, 670-678. The procedure will suitably be carried out utilising cultures of stably transfected eukaryotic cells, typically of stably transfected mouse Ltk⁻ fibroblast cells.

The triazolo-pyridazine derivative of the present invention exhibits anxiolytic activity, as demonstrated by a positive response in the elevated plus maze and conditioned suppression of drinking tests (cf. Dawson et al., Psychopharmacology, 1995, 121, 109-117). Moreover, the triazolo-pyridazine derivative of the invention is substantially non-sedating, as confirmed by an appropriate result obtained from the response sensitivity (chain-pulling) test (cf. Bayley et al., J. Psychopharmacol., 1996, 10, 206-213).

The triazolo-pyridazine derivative of the present invention may also exhibit anticonvulsant activity. This can be demonstrated by the ability to block pentylenetetrazole-induced seizures in rats and mice, following a protocol analogous to that described by Bristow et al. in J. Pharmacol. Exp. Ther., 1996, 279, 492-501.

Since it elicits behavioural effects, the triazolo-pyridazine derivative of the invention plainly is brain-penetrant; in other words, it is capable of crossing the so-called “blood-brain barrier”. Advantageously, the triazolo-pyridazine derivative of the invention is capable of exerting its beneficial therapeutic action following administration by the oral route.

The invention also provides pharmaceutical compositions comprising the glucuronide adduct of formula I as depicted above in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of the active ingredient. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. Typical unit dosage forms contain from 1 to 100 mg, for example 1, 2, 5, 10, 25, 50 or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

In the treatment of anxiety, a suitable dosage level is about 0.01 to 250 mg/kg per day, preferably about 0.05 to 100 mg/kg per day, and especially about 0.05 to 5 mg/kg per day. The compound may be administered on a regimen of 1 to 4 times per day.

The compound of formula I as depicted above is an unexpected human liver metabolite of the compound of formula II:

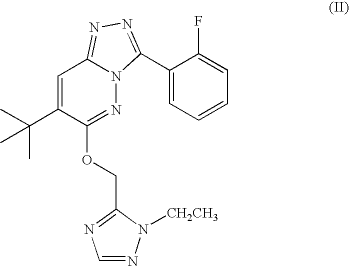

Thus, compound I can be isolated from an incubate of compound II above and human liver microsomes fortified with UDP-glucuronosyltransferase.

For commercial applications, the compound of formula I as depicted above may be prepared by a process which comprises reacting the compound of formula II as depicted above with the compound of formula III:

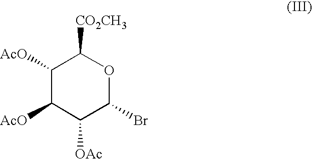

to afford the intermediate of formula IV:

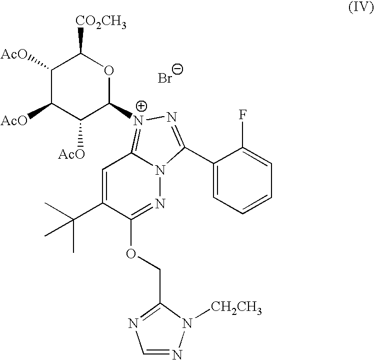

wherein Ac is an abbreviation for acetyl; then treating the intermediate of formula IV thereby obtained with a basic reagent to remove the acetyl protecting groups; followed by treatment with trifluoroacetic acid and isolation by preparative high-performance liquid chromatography (HPLC) to afford the intermediate of formula V:

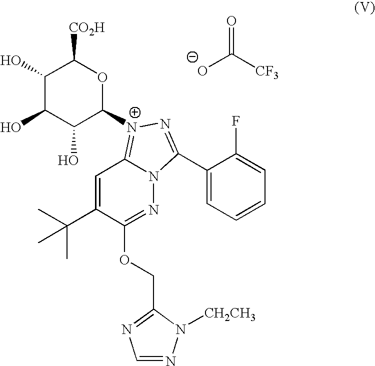

with subsequent desalting of the intermediate of formula V thereby obtained to provide the required zwitterionic product of formula I.

The reaction between compounds II and III above is an example of the Koenigs-Knorr synthesis. It is conveniently effected in the presence of a cadmium salt such as cadmium carbonate, suitably in a solvent such as nitromethane, typically at the reflux temperature of the solvent.

A suitable basic reagent which may be employed in removing the acetyl protecting groups from the intermediate of formula IV is sodium carbonate. The transformation is conveniently effected in a solvent such as an aqueous lower alkanol, e.g. aqueous methanol, typically at a temperature in the region of 0° C.

Desalting of the intermediate V may conveniently be achieved by preparative HPLC, eluting with a suitable solvent system, typically water/acetonitrile.

The compound of formula II above may be prepared by the procedures described in WO 99/67245, or by methods analogous thereto.

The following Example illustrates the preparation of the compound according to the invention.

The triazolo-pyridazine derivative in accordance with this invention potently inhibits the binding of [³H]-flumazenil to the benzodiazepine binding site of human GABA_{A} receptors containing the α2 or α3 subunit stably expressed in Ltk⁻ cells.

Reagents

Phosphate buffered saline (PBS).

Assay buffer: 10 mM KH₂PO₄, 100 mM KCl, pH 7.4 at room temperature.

[3H]-Flumazenil (18 nM for α1β3γ2 cells; 18 nM for α2β3γ2 cells; 10 nM for α3β3γ2 cells) in assay buffer.

Flunitrazepam 100 μM in assay buffer.

Cells resuspended in assay buffer (1 tray to 10 ml).

Harvesting Cells

Supernatant is removed from cells. PBS (approximately 20 ml) is added. The cells are scraped and placed in a 50 ml centrifuge tube. The procedure is repeated with a further 10 ml of PBS to ensure that most of the cells are removed. The cells are pelleted by centrifuging for 20 min at 3000 rpm in a benchtop centrifuge, and then frozen if desired. The pellets are resuspended in 10 ml of buffer per tray (25 cm×25 cm) of cells.

Assay

Can be carried out in deep 96-well plates or in tubes. Each tube contains:

300 μl of assay buffer.

50 μl of [³H]-flumazenil (final concentration for α1β3γ2: 1.8 nM; for α2β3γ2: 1.8 nM; for α3β3γ2: 1.0 nM).

50 μl of buffer or solvent carrier (e.g. 10% DMSO) if compounds are dissolved in 10% DMSO (total); test compound or flunitrazepam (to determine non-specific binding), 10 μM final concentration.

100 μl of cells.

Assays are incubated for 1 hour at 40° C., then filtered using either a Tomtec or Brandel cell harvester onto GF/B filters followed by 3×3 ml washes with ice cold assay buffer. Filters are dried and counted by liquid scintillation counting. Expected values for total binding are 3000-4000 dpm for total counts and less than 200 dpm for non-specific binding if using liquid scintillation counting, or 1500-2000 dpm for total counts and less than 200 dpm for non-specific binding if counting with meltilex solid scintillant. Binding parameters are determined by non-linear least squares regression analysis, from which the inhibition constant Kᵢ can be calculated for each test compound.

The triazolo-pyridazine derivative of the accompanying Example was tested in the above assay, and was found to possess a Kᵢ value for displacement of [³H]-flumazenil from the α2 and/or α3 subunit of the human GABA_{A} receptor of less than 1 nM.

### EXAMPLE

a) Methyl Ester IV

A mixture of compound II (723 mg, 1.83 mmol) and bromo-2,3,4-tri-O-acetyl-α-D-glucopyranuronic acid methyl ester III (1.45 g, 3.66 mmol) in nitromethane (45 ml) was treated with CdCO₃ (473 mg, 2.74 mmol) and heated at reflux. After 3.5 h the mixture was cooled to room temperature and filtered through celite. The filtrate was reduced on a rotary evaporator and the residue purified by flash chromatography on silica gel (THF/toluene/MeOH 4:5:1 to 5:3:2). The product-rich fractions were combined, concentrated by rotary evaporation, and crystallized from methylcyclohexane/THF/dichloroethane (2:1:1). The resulting solid was collected on a sintered glass funnel and dried in vacuo to provide 325 mg of the title compound IV (22% yield based on starting compound II). ¹H NMR (CD₃CN) δ 8.88 (1H, s), 7.91 (1H, t, J 7.3 Hz), 7.84 (1H, s), 7.81 (1H, m), 7.51 (1H, dd, J 7.3, 7.7 Hz), 7.46 (1H, dd, J 7.3, 7.7 Hz), 7.13 (1H, d, J 9.4 Hz), 5.84 (1H, t, J 9.4 Hz), 5.66 (1H, t, J 9.4 Hz), 5.61 (2H, d, J 2.1 Hz), 5.38 (1H, t, J 9.80 Hz), 5.04 (1H, d, J 10.2 Hz), 4.08 (2H, dq, J 3.0, 7.3 Hz), 3.68 (3H, s), 2.05 (3H, s), 2.00 (3H, s), 1.86 (3H, s), 1.50 (9H, s), 1.32 (3H, t, J 7.3 Hz).

b) Quaternary Ammonium N-Alucuronide Adduct I

The protected glucuronide IV (307 mg, 0.386 mmol) was suspended in methanol (10 ml) and cooled to 0° C. with an ice bath. 0.5M Aqueous Na₂CO₃ (1.55 ml, 0.77 mmol) was then added dropwise over 5 minutes resulting in a homogeneous pale yellow solution. The cooling bath was removed and after 3.5 h stirring the solution was diluted with water (12 ml) and the pH adjusted to 4.0 with 1% aq. trifluoroacetic acid. The solution was reduced to ½ volume on a rotary evaporator and freeze-dried. The resulting solid was loaded onto a YMC J-sphere ODS-H80 preparative HPLC column (30×250 mm) and eluted with 0.1% TFA-water/acetonitrile (linear acetonitrile gradient from 10-40%). Pure product-containing fractions were combined, reduced to ¼ volume on a rotary evaporator and freeze-dried affording the TFA salt V. Desalting was achieved by loading the material into water and eluting through a Hamilton PRP-1 preparative HPLC column (21×250 mm) with water/acetonitrile (linear gradient from 0-100% acetonitrile). The product-containing fractions were combined, concentrated to ¼ volume by rotary evaporation, and freeze-dried, affording 60 mg of the title compound (zwitterion I) as a white powder. ¹H NMR (CD₃OD) δ 8.72 (1H, s), 8.03 (1H, t, J 8.0 Hz), 7.99 (1H, s), 7.80 (1H, m), 7.49 (2H, dt, J 8.0, 14.9 Hz), 6.32 (1H, d, J 8.9 Hz), 5.72 (2H, s), 4.32 (1H, d, J 9.3 Hz), 4.21 (3H, m), 3.74 (2H, dt, J 9.0, 21.0 Hz), 1.50 (9H, s), 1.40 (3H, t, J 7.2 Hz). LC-MS (M+H; expected 572.23, observed 572.25).

1-9. (canceled)..

10. A compound of the formula I: 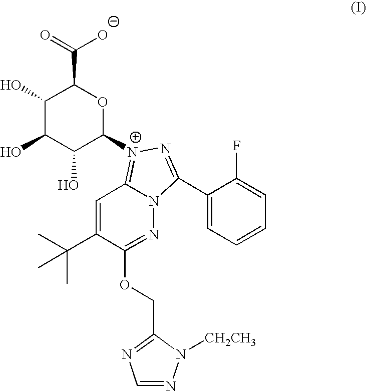
or a pharmaceutically acceptable salt thereof.

11. The compound of claim 10 or a pharmaceutically acceptable salt thereof in isolated form.

12. A pharmaceutical composition comprising the compound of claim 10 or a pharmaceutically acceptable salt thereof and a pharmaceutically acceptable carrier.

13. A compound of the formula I:

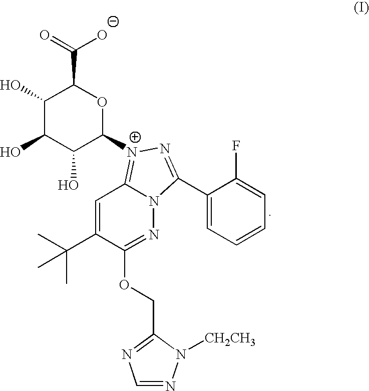

14. The compound of claim 13 in isolated form.

15. A pharmaceutical composition comprising the compound of claim 13 and a pharmaceutically acceptable carrier.

16. A process for the preparation of the compound of claim 10 which comprises reacting the compound of formula II: 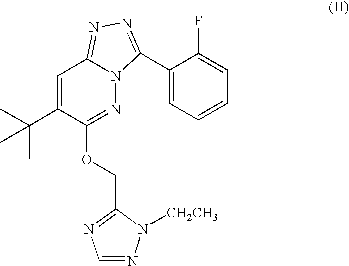
with the compound of formula III: 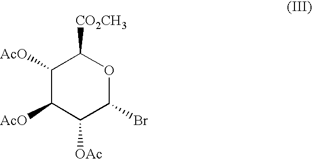
to afford the intermediate of formula IV: 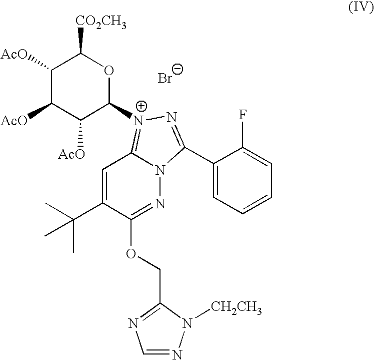
wherein Ac is an abbreviation for acetyl; then treating the intermediate of formula IV thereby obtained with a basic reagent to remove the acetyl groups; followed by treatment with trifluoroacetic acid and isolation by preparative high-performance liquid chromatography (HPLC) to afford the intermediate of formula V: 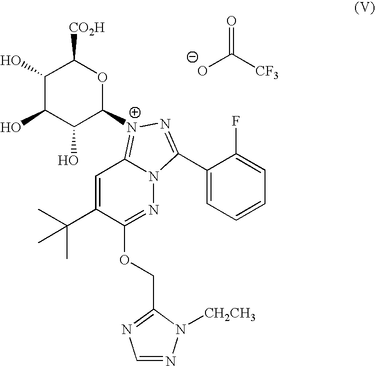
followed by desalting of the intermediate of formula V thereby obtained to provide the compound of claim 1.

17. A method for the treatment of anxiety which comprises administering to a patient in need of such treatment an effective amount of the compound of claim 10.

18. A method for the prevention of anxiety which comprises administering to a patient in need of such prevention an effective amount of the compound of claim 10.

19. A method for the treatment of convulsions which comprises administering to a patient in need of such treatment an effective amount of the compound of claim 10.

20. A method for the prevention of convulsions which comprises administering to a patient in need of such prevention an effective amount of the compound of claim 10.
